(19) United States
(12) Patent Application Publication
(10) Pub. No.: US 2015/0047666 A1
(43) Pub. Date: Feb. 19, 2015

(54) PERMANENT WAVE TREATMENT METHOD

(71) Applicant: TAKARA BELMONT CORPORATION, Osaka (JP)

(72) Inventors: Motoo SAKURAI, Osaka-shi (JP); Kazuya YAMANAKA, Osaka-shi (JP); Terumi SHIBANO, Osaka-shi (JP); Reiko NAKASHIMA, Osaka-shi (JP)

(73) Assignee: TAKARA BELMONT CORPORATION, Osaka (JP)

(21) Appl. No.: 13/969,379

(22) Filed: Aug. 16, 2013

(51) Int. Cl. A61K 8/49; A45D 7/04; A45D 7/06; A61Q 5/04

(52) U.S. Cl. CPC A61K 8/4973; A61Q 5/04; A45D 7/04; A45D 7/06
USPC 132/206; 132/207

(57) ABSTRACT

A permanent wave treatment method is provided in which the difference between wet and dry wave shape is small.

A first permanent wave agent is applied to swell and soften hair wound around a rod, the first permanent wave agent is rinsed off, a chemical agent spray member 42 is moved back and forth inside a hood 2 while uniformly spraying the entire hair with a chemical agent comprising an intermediate treatment agent that contains ascorbic acid salt; while heating with a blower 12, the ascorbic acid salt is adhered to the hair wound around the rod, and the moisture is maintained. A negative pressure state is formed in the rod, and while the hair are being dried by the blower 12, ozone from an ozone unit 3 is sprayed into the hood 2, forming an ozone atmosphere that envelopes the entire hair, oxidizing the ascorbic acid salt on the hair wound around the rod, forming dehydroascorbic acid salt, the oxidized action of which is exerted then and there, promoting recoupling of disulfide bonds in the entire hair enveloped in the ozone atmosphere.

### FIELD OF THE INVENTION

The present invention relates to a permanent wave treatment method that can suppress damage to the hair, reduce the difference between the wet and dry wave shape, and improve work efficiency of a salon by reducing the time a practitioner is tied up by the work.

### BACKGROUND ART

In patent document 1 below, the present applicant proposed a permanent wave treatment method that enabled an initial wave that was close to the diameter of a rod to be maintained for an extended period. The invention comprises treatment with a first permanent wave agent followed by an intermediate washing, stopping the progress of the open chain of the disulfide bonds, arranging the hair in a state suitable for the rearrangement of hair molecules (polymer movement), transition of the hair into a dry state, and treatment by a second permanent wave agent, to recouple the disulfide bonds to form a wave of a desired shape.

The permanent wave treatment method comprises a first step for winding the hair around a plurality of rods, a second step for applying to the wound hair a first permanent wave agent containing a reducing agent to swell and soften the hair, a third step comprising rinsing off the first permanent wave agent adhering to the swelled, softened hair, or stopping the action of the first permanent wave agent by using an intermediate treatment agent that neutralizes the first permanent wave agent, or both the rinsing off and the stopping the action, a fourth step in which, following the third step, the subject person's head portion is covered by the hood of a permanent wave treatment device and left for a set time while a blower is driven to heat the hair, maintaining a moist state, or with the whole of the subject person's head, from the forehead to the back of the head, being covered by a cloth, and the blower is driven on a low setting, to create a negative pressure in the rods to maintain a moist state, a fifth step for drying the hair with a negative pressure in the rods while driving the blower on high, and a sixth step in which, after the hair is dried, a second permanent wave agent containing an oxidizing agent is applied and left for a set time to recouple the disulfide bonding to memorize the dry-state shape.

### DESCRIPTION OF THE PRIOR ART

### Patent Documents

[Patent document 1] Japanese Patent No. 4473848

### DISCLOSURE OF THE INVENTION

However, when the invention thus disclosed by the above patent document 1 is practiced, during the steps for arranging the hair preferably for rearranging the molecules thereof, drying the hair and then treating the hair with the second permanent wave agent, the subject person is forced to maintain the same posture on the cutting chair, which is burdensome to the subject person. Also, during the treatment with the second permanent wave agent, the practitioner cannot do any other work at the same time as he or she has to stay with the one subject person, reducing the salon work efficiency. There is a need to improve this. Another problem is that as the second permanent wave agent is a water-soluble liquid, in a wet environment the recoupling of the disulfide bonds progresses in a direction that weakens the hair shape formed in the drying step, so that when the hair dries the wave shape results in different from when the hair is wet.

The present invention has been proposed in view of the above situation. It is an object of the present invention to provide a permanent wave treatment method that can reduce the difference between wet and dry wave shape, shorten the amount of time the practitioner is occupied by the treatment, thereby improving the work efficiency of the salon, alleviate the burden on the subject person and suppress damage to the hair.

To attain the above object, the present invention characterized in that it comprises applying a first permanent wave agent having a reduction action to swell and soften hair wound around a rod, rinsing off the first permanent wave agent, applying to the hair one, two or more compounds selected from a group consisting of ascorbic acid, ascorbic acid derivative and ascorbic acid salt, and an intermediate treatment agent, then spraying ozone at the hair to oxidize the compound, and recoupling disulfide bonds of the hair by the oxidized compound and the ozone.

Furthermore, in the above permanent wave treatment method it is preferable that the intermediate treatment agent contains a concentration of 0.13 to 24 percent by weight, and an ozone atmosphere is formed containing 1.0 or less pm of the ozone.

In particular, in the recoupling of the disulfide bonds of the hair it is also preferable that the compound is adhered to the hair wound around the rod and the moisture of the hair is maintained while heating is applied for a prescribed time, after which a negative pressure state is established inside the rod, and while a blower is being used for drying, ozone is sprayed towards the hair to envelop the hair in an ozone atmosphere. In the present invention, the oxidized compound is dehydroacid or a dehydroascorbic acid salt.

The above permanent wave treatment method also comprises a second permanent wave agent having an oxidizing action being applied to the hair in which disulfide bonds have been recoupled, for further recoupling of disulfide bonds.

In the permanent wave treatment method according to the present invention, one, two or more compounds selected from a group consisting of ascorbic acid, ascorbic acid derivative and ascorbic acid salt, and an intermediate treatment agent, are applied to the hair, following which ozone is sprayed on the hair to oxidize the compound, whereby disulfide bonds of the hair are recoupled by the oxidized compound and the ozone. Therefore, the disulfide bonds of the hair can be recoupled in a dry ozone environment by the oxidized compound and the ozone, thereby resolving the problem of the difference between the wet and dry wave shape wherein a wave shape of dried hair differs from that in the treatment process. Since the combined use of the oxidized compound and the ozone exerts a better oxidizing action, a permanent wave having a good finish can be formed. Also, recoupling the disulfide bonds in a dry environment enables the treatment components of the intermediate treatment agent to fully permeate the hair. When along with the intermediate treatment agent a compound such as ascorbic acid or the like applied to the hair is oxidized by ozone, it forms dehydroascorbic acid or a dehydroascorbic acid salt, the oxidizing action thereof can be promptly utilized to effectively form a permanent wave having a good finish.

The second permanent wave agent not having to be applied and left for a fixed time frees up the practitioner, thereby improving the salon work efficiency and, by also shortening the overall treatment operation time, greatly reducing the burden on the subject person. Since the oxidation effect of the ozone and the oxidized compound can be promptly exerted on the hair, hair damage caused by residual oxidizing action, which is a concern, can be reduced. Demonstrating the reducing action provided by the ascorbic acid salt, the drying and oxidizing steps can be started without any impediment to the rearranging of hair molecules.

In the present invention, in particular, permanent waves can be formed having a better finish due to the intermediate treatment agent containing a compound concentration of 0.13 to 24 percent by weight, and the ozone atmosphere formed containing not more than 1.0 ppm of ozone. Moreover, as the oxidation effect can be efficiently obtained under the above compound and ozone parameters, the amount of ozone needed to recouple the disulfide bonds can be reduced, which ensures the safety and comfort of practitioner and subject person and makes it possible to improve the safety and comfort of the treatment procedure.

In the present invention, also, in the recoupling of the disulfide bonds of the hair, the compound is adhered to the hair wound around the rod and the moisture of the hair is maintained while heating is applied for a prescribed time. Therefore, the oxidizing action of the dehydroascorbic acid or dehydroascorbic acid salt that is the compound oxidized by ozone can be effectively extended to the hair wound on the rod. Assuming that the oxidizing action of the oxidized compound effectively exerts, the amount of ozone required can be further reduced. At the same time, the hair can be arranged in a state suitable for the rearrangement of the hair molecules (polymer movement). In addition, the invention establishes a negative pressure state inside the rod, so that while the hair is being dried by the blower, an ozone mist is sprayed towards the hair, enveloping the hair in an ozone atmosphere. The compound and ozone can thus be efficiently brought into contact with the hair wound on the rod, ensuring the secure progression of the disulfide bond recoupling in a dry environment, further resolving the wet-dry difference. Having the hair enveloped in an ozone atmosphere ensures that the oxidizing action is applied uniformly to suppress unevenness in the finish.

Further recoupling of disulfide bonds of hair, wherein disulfide bonds have been recoupled by the oxidized compound and the ozone, can be done by applying to the hair a second permanent wave agent having an oxidizing action. This operation of applying the second permanent wave agent can be completed in the very short time of 1 to 3 minutes. The step can be included in the treatment procedure carried out at the shampoo basin, where it requires substantially no process time. An even more uniform finish can be obtained by this application of the second permanent wave agent to hair in which disulfide bonds have been recoupled by the oxidized compound and the ozone.

### BRIEF DESCRIPTION OF THE DRAWINGS

FIG. 1 is an overall perspective view of a permanent wave treatment device utilized to implement the permanent wave treatment method of the present invention.

FIG. 2 is a flowchart illustrating the overall permanent wave treatment method of the invention and a program mode settings.

FIG. 3 is a flowchart of a chemical agent spray mode in the permanent wave treatment method of the invention.

FIG. 4 is a flowchart of a creep mode in the permanent wave treatment method of the invention.

FIG. 5 is a flowchart of a glass transition mode in the permanent wave treatment method of the invention.

### DESCRIPTION OF THE EMBODIMENTS

The permanent wave treatment method according to the present invention will now be described in detail in the following. The embodiment described below is one example of a specific implementation of the invention, which is not limited thereto. The design may be modified in various ways so long as such modifications do not depart from the scope of the invention as described in the scope of patent claims.

The present invention relates to a permanent wave treatment method in which a first permanent wave agent having a reducing action is applied to hair wound around a rod to swell and soften the hair, the first permanent wave agent is rinsed off and an intermediate treatment agent is applied, following which disulfide bonds of the hair are recoupled. The intermediate treatment agent contains 0.13 to 14 percent by weight (0.01 to 1.87 M) of one, two or more compounds selected from a group consisting of ascorbic acid, ascorbic acid derivative, and ascorbic acid salt. Instead of being contained in the intermediate treatment agent itself, the one, two or more compounds selected from a group consisting of ascorbic acid, ascorbic acid derivative, and ascorbic acid salt may be prepared in the salon directly before use as a compound powder or the like adjusted to be 0.13 to 24 percent by weight of the intermediate treatment agent. In such a case, a technique may be proposed in which the prepared compound is applied to the hair and the intermediate treatment agent is also applied to the hair.

When recoupling the disulfide bonds of the hair, an ozone atmosphere containing not more than 1.0 ppm ozone is formed in which the hair is contacted with ozone mist. The ozone oxidizes the one, two or more compounds selected from a group consisting of ascorbic acid, ascorbic acid derivative, and ascorbic acid salt that have already been applied to the hair, and the disulfide bonds in the hair are recoupled by the oxidized compound and the ozone. In particular, when recoupling the disulfide bonds of the hair, heating is applied for a prescribed time (3 to 15 minutes, for example) to ensure that the compound, such as ascorbic acid or the like, contained in the intermediate treatment agent adheres to the hair wound around the rod. At this time, the moistness of the hair is maintained. In addition, a negative pressure state is established inside the rod, and while the blower is being used for drying, ozone is sprayed at the hair to form an ozone atmosphere so that all of the hair are enveloped in a uniform concentration of ozone, and the compound and ozone can be made to efficiently contact the hair wound around the rod.

In the invention, recoupling of the disulfide bonds proceeds by means of the oxidized compound (dehydroascorbic acid and so forth) and ozone in the dry environment of the ozone atmosphere. Therefore, with the treatment process in which the second permanent wave agent is applied in a conventional wet environment being followed by the finish with the dry hair, the problem of the difference between wet and dry wave shape is resolved. Moreover, since with the formation of the ozone atmosphere the entire hair is enveloped by a uniform concentration of ozone, the oxidizing action is uniformly manifested over the entirety, suppressing unevenness in the permanent wave finish. When the ascorbic acid or the like compound applied to the hair together with the intermediate treatment agent is made to securely adhere to the hair wound around the rods and is oxidized by ozone to form dehydroascorbic acid or dehydroascorbic acid salt, its oxidizing action is exerted then and there due to its labile properties, efficiently extending the action to the hair wound on the rod. That is, the efficiency of the oxidizing effect is increased by the combined use of the oxidized compound and ozone, keeping down the amount of ozone required (ozone mist concentration).

In the present invention in order to further suppresses the unevenness in the wave shape finish, an operation may be added, that is, a second permanent wave agent having an oxidizing action is applied to hair in which disulfide bonds have been recoupled, for further recoupling disulfide bonds. This operation can be completed in the very short time of just 1 to 3 minutes. As the aim of this operation is to further suppress unevenness in the finish, the step can be included in the treatment procedure carried out at the shampoo basin, where the second permanent wave agent application process time is substantially zero.

The concentration of the one, two or more compounds selected from a group consisting of ascorbic acid, ascorbic acid derivative, and ascorbic acid salt is 0.01 to 1.87 M (0.13 to 24 percent by weight) with respect to the intermediate treatment agent. The use of a concentration that is lower than 0.01 M (0.13 percent by weight) is not recommended, since even if dehydroascorbic acid or dehydroascorbic acid salt is produced by oxidation by the ozone, the oxidizing effect is unlikely to extend to the hair. A concentration that is higher than 1.87 M (24 percent by weight) also is not recommended, as in addition to the risk of saturation of the oxidizing effect of the ascorbic acid itself and the bleaching action of the ascorbic acid, there may be other adverse effects, such as unevenness in the finished wave texture and shape.

The reason for the concentration of the ozone mist contacting the hair and forms the ozone atmosphere is set at not more than 1.0 ppm is safety considerations with respect to ozone sprays and the human body. The lower limit of the concentration used to form the ozone atmosphere may be set at a suitable level, such as 0.05 ppm, for example, based on considerations related to the desired wave shape finish in the permanent wave treatment, unevenness, and so forth.

### Examples

An embodiment of the permanent wave treatment method according to the present invention will now be described with reference to the drawings. The embodiment was implemented using a new permanent wave treatment apparatus having a configuration based on the present invention.

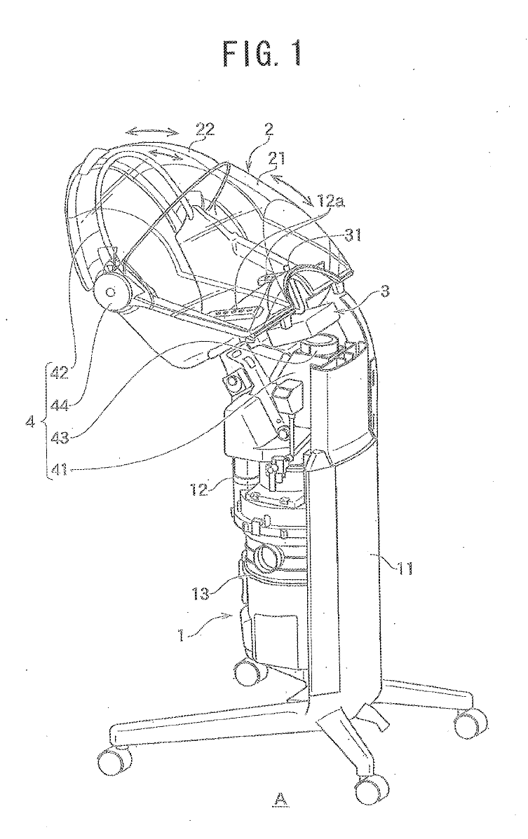

First the permanent wave treatment apparatus will be described. With reference to FIG. 1, the permanent wave treatment apparatus A comprises an apparatus body 1, a hood 2, an ozone unit 3 and a chemical agent spray unit 4, and can be moved freely by means of casters provided on the lower part.

The apparatus body 1 has a housing 11 that contains a circulatory air blower 12 consisting of a motor and fan that blows hot air heated by a heater into the hood 2, suctions air from inside the hood 2 and reheats the air, a moisture separator 13 that separates moisture and extraneous matter produced in the permanent wave treatment from the hot air suctioned by the circulatory air blower 12, and an atmospheric air introducing device (not shown) that introduces atmospheric air and mixes it with hot air blown out into the hood 2 to cool it.

The blower 12 blows hot air into the hood 2 and suctions air from the hood 2 via a hot air blowout port (not shown) and a suction port 12a each formed in the housing 11. The hot air blown into the hood 2 is sucked from a rod around which hair is wound, and to the suction port 12a via a tube attached to the rod and the suction port 12a. Considering the hot air flow in the hood 2, the preferable configuration is that the suction port 12a is formed at both sides in the upper part of the housing 11, and the hot air blowout port (not shown) is formed in the region between the suction ports 12a in the upper part of the housing 11. In order to make the temperature in the hood 2 uniform, and the ozone concentration in the ozone atmosphere uniform described below, it is also the preferable configuration that the hot air blowout port region has a prescribed area (for example 5 to 50 times the area of the suction ports 12a) and is formed in the upper part of the housing 11 between the suction ports 12a.

In the housing 11, a pipeline that runs from the heater to the hot air blowout port has a branch port from which a pipe is connected to the atmospheric air introducing device. Via the branch port, atmospheric air is mixed with the hot air by driving a fan provided on the atmospheric air introducing device. An atmospheric air intake port for taking air into the atmospheric air introducing device is formed on the side of the housing 11. Numerous air flow holes are formed in the circumferential surface of the rod. The rod is connected to the suction ports 12a by tubes, so suction by the blower 12 produces a negative pressure state in the tube, creating a negative pressure state in the rod.

The hood 2 is comprised of two separate hoods, a first hood 21 and a second hood 22, so as to be openable. The first hood 21 and the second hood 22 are coupled to the upper part of the housing 11 by a coupling part. By means of the coupling part, the first hood 21 can be rotated in a direction in which it overlaps the second hood 22. The second hood 21 is provided at the other side of the first food and, by means of the coupling part, can be rotated in a direction in which it overlaps the second hood 22. The first and second hoods 21 and 22 can be any shape that allows them to cover a head portion, such arc-shaped, square, hexagonal, and so forth.

The ozone unit 3, provided on the upper part of the housing 11, is an ozone generator that generates ozone from atmospheric air oxygen, using the electrical discharge or ultraviolet radiation method or the like. The front of the ozone unit 3 has an ozone blowout port 31 that protrudes through the first hood 21 into the hood 2, and can blow generated ozone into the hood 2. The ozone blowout port 31 is located in the vicinity of the hot air blowout port. While the ozone blowout port 31 shown in FIG. 1 is round, it can be another shape such as arc-shaped, rectangular, or a flattened oval shape or the like. The shape selected for the ozone blowout port 31 should be one that allows ozone blown from the port to be diffused in the hood 2 by efficiently riding the hot air blown from the hot air blowout port.

That is, an ozone atmosphere having a uniform concentration is formed in the hood 2 by the ozone generated by the ozone unit 3 efficiently riding the hot air blown out by the relatively large area of the nearby hot air blowout port. The ozone diffusion state can be used to form an ozone atmosphere with a uniform concentration of not more than 1.0 ppm in the hood 2. If the supply of ozone from the ozone unit 3 is shut off, the ozone in the hood 2 rapidly autolyzes and disappears. The amount of ozone supplied by the ozone unit 3 can be controlled to suitably regulate the concentration of the ozone atmosphere in the hood 2.

The chemical agent spray unit 4 is comprised of a chemical agent atomization kettle 41 that accommodates a chemical agent that is the intermediate treatment agent containing the one, two or more compounds selected from the group consisting of ascorbic acid, ascorbic acid derivative and ascorbic acid salt and that has an atomizing structure that utilizes ultrasound or the like to atomize the chemical agent, a chemical agent spray member 42 that moves inside the hood 2 so that hair is uniformly misted by the chemical agent atomized by the chemical agent atomization kettle 41, a fan 43 that blows the atomized chemical agent from the chemical agent atomization kettle 41 to the agent spray member 42, and a motor 44 that drives the agent spray member 42 back and forth along the inside surface of the hood 2.

The chemical agent spray member 42 has a curved cylindrical shape that corresponds to the inside shape of the hood 2, and a plurality of misting holes formed in the surface thereof. As shown in FIG. 1, the back and forth motion of the member 42 along the inside surface of the hood 2 is achieved by means of the motor 44 incorporated in the coupling part of the hood 2, using a gear mounted on the motor shaft and a corresponding gear attached to the end part of the member 42. In addition to its back and forth movement along the inside surface of the hood 2 (left and right, in FIG. 1), the spray member 42 is capable of making complex moves along the inside surface of the hood 2 if it is equipped with a plurality of joints or the like.

The chemical agent is an intermediate acid rinse treatment agent that contains 0.13 to 24 percent by weight of one, two or more compounds selected from the group consisting of ascorbic acid, ascorbic acid derivative and ascorbic acid salt. The spray amount thereof may be suitably adjusted in accordance with the amount of hair to be given the permanent wave treatment, and hair properties such as hardness or softness or the like. When the intermediate treatment agent does not contain the one, two or more compounds selected from the group consisting of ascorbic acid, ascorbic acid derivative and ascorbic acid salt, the compound is prepared in the salon directly before use as a powder or the like adjusted to be 0.13 to 24 percent by weigh, and after the compound is applied to the hair, the chemical agent spray unit 4 is operated to spray the hair with the intermediate treatment agent not containing the compound.

Although not shown, the permanent wave treatment apparatus A has a control section with an operation display screen and CPU that displays time, temperature, number of rods, amount of hair, hair damage, ozone spray device operation, spay of chemical agent, and so forth. The control section also has buttons for setting each display. When the practitioner operates the buttons to make settings, information is processed by the CPU and the various types of controls in the permanent wave treatment apparatus A are executed. Specifically, there are an ozone setting button that controls the ozone concentration level by, for example, controlling the current to the ozone unit 3; a spray amount control setting button that controls the amount of chemical agent sprayed by the chemical agent spray unit 4a; a motor setting button that controls the back and forth movement of the chemical agent spray member 42 on the inside surface of the hood 2; a button for setting the number of rods around which a subject person's hair is wound; a button for setting the amount of the subject person's hair; a button for setting the checking of the state of damage to the subject person's hair, and so on. Operating each button causes the corresponding treatment time and treatment temperature data to be stored in ROM in the control section and the data is processed by the CPU. If the practitioner wishes, the various settings can be stored in RAM in the control section.

Control section controls by the practitioner operating the setting buttons include controlling the heater temperature by switching the heater on and off, controlling times by means of a timer that calculates the times set by the ROM, and controlling the blower hot air flow and temperature by switching it on and off, controlling the blower for the amount of air flow, and so forth. The control section also has a start switch for starting these controls. When the hood 2 has a sensor to detect the temperature in the hood 2, the control section also controls the temperature in the hood 2 and has a switch for starting the temperature control. Preferably, the control section should also have a safety button or the like for switching the power off if anything unusual happens. The practitioner operates the various switches to have the permanent wave treatment apparatus A execute a series of permanent wave treatments.

A series of permanent wave treatments using the above permanent wave treatment apparatus A will now be described, with reference to an example in which the compound in the intermediate treatment agent is ascorbic acid salt.

The practitioner winds the subject person's hair around the rod and applies to the hair a publicly known first permanent wave agent having a reducing action, and leaves it in that state for a prescribed time to swell and soften the hair. After confirming the hair has swelled and softened, the first permanent wave agent adhering to the hair is rinsed off, the hair is toweled dry, and the subject person is seated at the permanent wave treatment apparatus A. It is convenient if at this point the hood 2 has been left open by rotating the first and second hoods 21 and 22 so they overlap. Once the subject person is seated, the hoods 21 and 22 are rotated to close the hood 2 and cover the subject person's head portion. The open side of the second hood 22 corresponding to the subject person's face side is covered with a cloth, creating a condition in which the inside of the hood 2 is sealed so hot air cannot leak out. In this state, the practitioner operates the various setting buttons of the permanent wave treatment apparatus A to implement the permanent wave treatment method of the present invention.

Firstly, the chemical agent spray unit 4 is operated to move the chemical agent spray member 42 in the hood 2 while uniformly spraying the chemical agent over the entire hair. The concentration of the ascorbic acid salt in the chemical agent at this time is suitably set from the relationship with the ozone concentration of the ozone atmosphere described below; however, a reasonable concentration is 0.05 to 0.72, and for cost reasons, a concentration of 0.05 to 0.36 M is recommended. The treatment effect is imparted to the hair by adhering the chemical agent on the hair. Demonstrating the little reducing action provided by the ascorbic acid salt, the drying/oxidizing step can be started without hindering the rearrangement of the hair molecules. The practitioner checks to see if the chemical agent is adhering to the portion of the hair wound around the rear of the rod, with respect to the agent spray member 42. If he or she sees the spraying is uneven, operation of the chemical agent spray unit 4 can be localized for fine adjustment to ensure the chemical agent adheres uniformly over the whole of the hair, or this can be done manually.

The heating by the blower 12 is continued and the moisture maintained for a prescribed time (for example, around 3 to 15 minutes) controlled in accordance with the hardness and amount of hair and the like, allowing further adhesion of the chemical agent in the hair wound around the rod. The blower 12 is controlled to blow out hot air at the controlled temperature and controlled strength (blowing weak or strong) so that the temperature and the atmosphere in the hood 2 is constant. In that state, the hair is heated only at the prescribed temperature (preferably 60 degrees or less) and time (which differs depending on the hair hardness and amount and the like), forming a suitable state for the rearrangement (polymer movement) of the molecules in the hair.

As well as being heated by hot air from the blower 12, the hair may be heated by a steam or mist atmosphere using suitable means. When steam or mist is used for the heating, the amount of water for moist hair and the temperature can be readily controlled to be within desirable range, failures are few, and it allows permanent wave treatment to proceed with high wave efficiency. As described above, the permanent wave treatment apparatus A incorporates the various temperature and time control means, so automation is possible. Manual control can also be used to maintain the moisture of the hair.

Thereafter, by connecting the tube to the rod around which the hair are wound and to the suction port 12a, the blower 12 can be used to create a negative pressure in the rod, and ozone from the ozone unit 3 can be sprayed into the hood 2 to envelop the entire hair in an ozone atmosphere in which recoupling of the disulfide bonds in the hair can proceed. Hot air generated by the blower 12 is blown out from a region that occupies a relatively large area on the upper part of the housing, and as the hood 2 is in a sealed state, the entire hair is enveloped by ozone having a uniform concentration. The negative internal pressure of the rod therefore raises the efficiency with which the ozone contacts the hair, enabling the oxidizing action of the ozone to be efficiently utilized. The ozone mist can form an ozone atmosphere within the hood 2 having an ozone concentration 1.0 ppm or less. However, this can be suitably adjusted by the relationship with the ascorbic acid and the like contained in the chemical agent.

Ascorbic acid salt on the hair is oxidized by the ozone atmosphere in the hood 2, forming dehydroascorbic acid salt, the oxidizing action of which is exerted then and there, promoting the recoupling of disulfide bonds of hair to which it adheres. In addition, the oxidizing action of the ozone itself advances the recoupling of the disulfide bonds of the entire hair enveloped by the ozone atmosphere. The hair is dried by the blower 12, so the recoupling of the disulfide bonds progresses in a dry environment. Furthermore, as the oxidation progresses in an ozone atmospheric environment, the action extends uniformly to the entire hair.

Finally, after the practitioner has checked that the entire hair is sufficiently dried, he or she removes the rod from the hair to check the finish. Then, the subject person is given the treatment procedure at the shampoo basin. To further suppress unevenness in the finish, a publicly known second permanent wave agent having an oxidizing action may applied to hair in which disulfide bonds have been recoupled for further recoupling of disulfide bonds. This operation can be completed very quickly, taking just 1 to 3 minutes. Since such application of the second permanent wave agent is included in the treatment procedure, substantially no process time is involved. The procedure completes by drying and stylizing the hair.

The advantage of the present invention compared to the permanent wave treatment method disclosed in patent document 1 will now be described.

In the permanent wave treatment method according to patent document 1, before applying a second permanent wave agent and recoupling the disulfide bonds of the hair, the hair is heated, dried and arranged into a state suitable for rearrangement (polymer movement) of molecules in the hair to improve the wave efficiency. However, because the disulfide bond recoupling caused by the second permanent wave agent progresses under a wet environment, giving rise to the problem of the difference between the wet and dry wave shape wherein a wave shape of dried hair differs from that in the treatment process. Whereas, in the case of the present invention, the hair is heated and dried, providing improved wave efficiency, in addition to which, with the combined use of dehydroascorbic acid or the like and ozone, recoupling of the disulfide bonds proceeds in a dry environment, successfully resolving the problem of wet/dry differences. Also, with the entire hair being enveloped in the ozone atmosphere within the sealed hood, the oxidizing action is manifested uniformly entirely therein, enabling unevenness in the permanent wave finish to be suppressed. The combined use of a dehydroascorbic acid salt or the like makes it possible to keep down the amount of ozone required for the mist (the concentration of the ozone sprayed into contact). This enables the ozone concentration to which the practitioner and the subject person are exposed to be kept to within the permissible concentration level recommended by the Japan Society for Occupational Health, thus ensuring their safety and comfort and raising the safety and comfort of the treatment procedure.

In particular, in the permanent wave treatment method of the patent document 1, the subject person is forced to maintain the same posture on the cutting chair during the steps of heating and drying the hair, and treating the hair with the second permanent wave agent, which is very burdensome for the subject person. In contrast, in the case of the present invention, recoupling of the disulfide bonds takes place during the steps of heating and drying the hair, so in practice the treatment step (operation time) using the second permanent wave agent can be omitted, thereby greatly reducing the burden on the subject person. This also frees up the practitioner, which should provide a major increase the salon's work efficiency.

In cases in which a publicly known second permanent wave agent having an oxidizing action is applied to hair in which disulfide bonds have been recoupled, to effect more recoupling of disulfide bonds to further suppress unevenness in the finish, this can be included in the treatment procedure done on the subject person at the shampoo basin, so that substantially no process time is required. That is, the present invention succeeds in advancing permanent wave treatment that provides a better finish without reducing work efficiency in the salon.

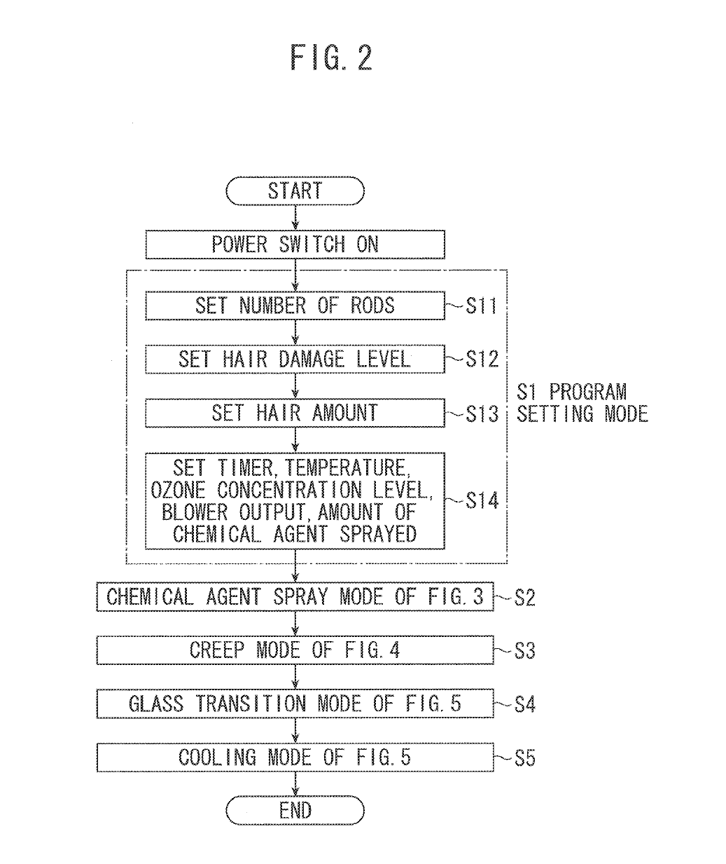

With respect to the permanent wave treatment method described in the foregoing, a supplementary explanation based on the flow charts of FIGS. 2 to 4 is provided here relating to the processes that take place after the permanent wave treatment apparatus A is operated.

With reference to FIG. 2, when a power switch is switched on to supply power from an external alternating current source, the practitioner operates the various setting buttons on the control section, the procedure moves on in the following order: program setting mode S1, for program settings; chemical agent spray mode S2, for applying an intermediate treatment agent containing a compound to the hair; creep mode S3 (drying step) for adhering a chemical agent (compound) and maintaining hair moisture and the like; glass transition mode S4 (oxidation step) in which recoupling of disulfide bonds in the hair is enhanced by contacting a compound oxidized with an ozone mist and ozone and cooling mode S5 for cooling the hair.

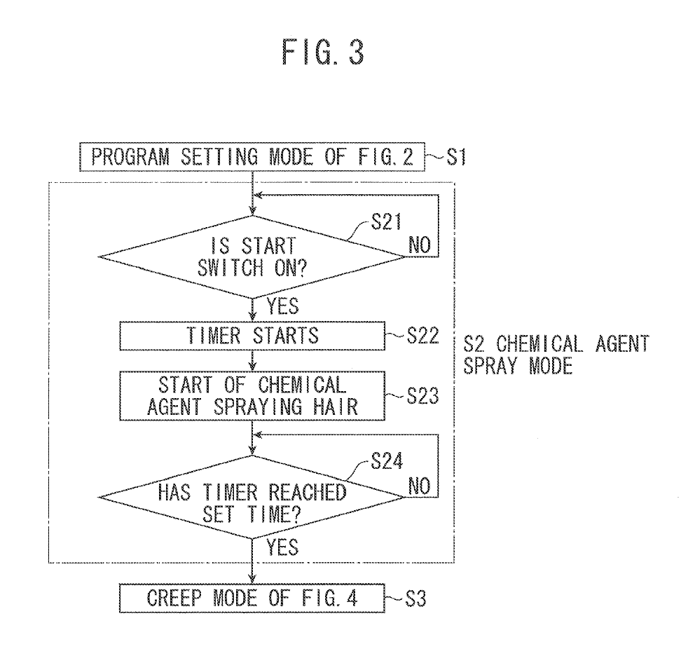

First, in program setting mode S1, the practitioner uses the various switches on the control section to make the program settings, such as the number of rods (step S11), hair damage level (step S12), and amount of hair (step S13). When this is done, the timer, temperature, blower output, ozone concentration and level, and the chemical agent spray amount, including the movements of the chemical agent spray member 42, each corresponding to the conditions set by the control section, are read out of ROM and the modes set (step S14). The CPU incorporated in the control section moves to the chemical agent spray mode S2 shown in FIG. 3, and monitors whether or not the practitioner has turned on the switch for starting the chemical agent spray (step S21). If it is determined that the switch has been turned on, the timer set in program setting mode S1 starts (step S22).

Simultaneously with the start of the timer in step S22, the chemical agent spray member 42 of the permanent wave treatment apparatus A is used to blow a controlled amount of chemical agent mist at the hair (step S23). Because the spray amount is set by the hair amount, damage level, and number of rods set in program setting mode S1, the adhesion amount of the chemical agent is also optimum.

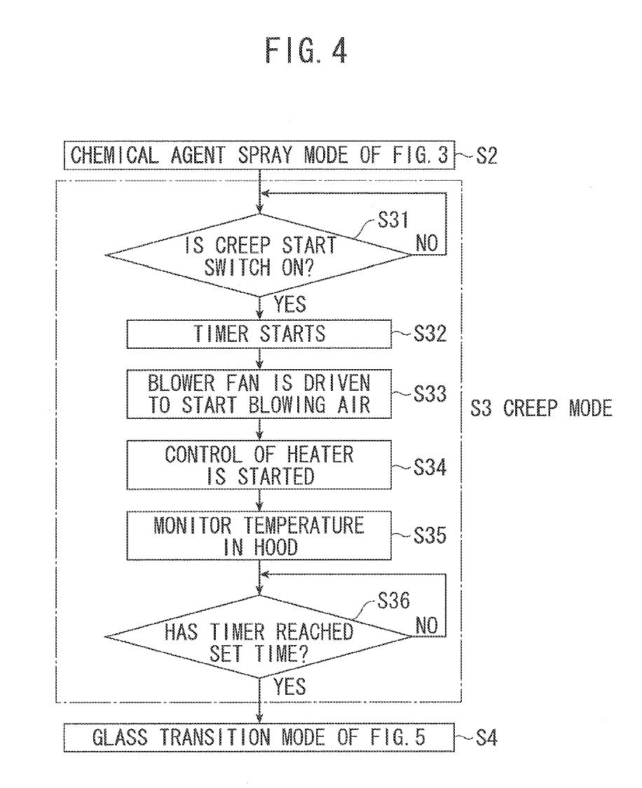

Next, the CPU monitors whether or not the timer started in step S22 has reached the set time (step S24). If it is determined that the set time has been reached, the procedure moves to creep mode S3 shown in FIG. 4, and the CPU monitors whether or not the practitioner has switched on the creep start switch (step S31). If it is determined that it has been switched on, the timer starts at the setting set in program settings mode S1 (step S32).

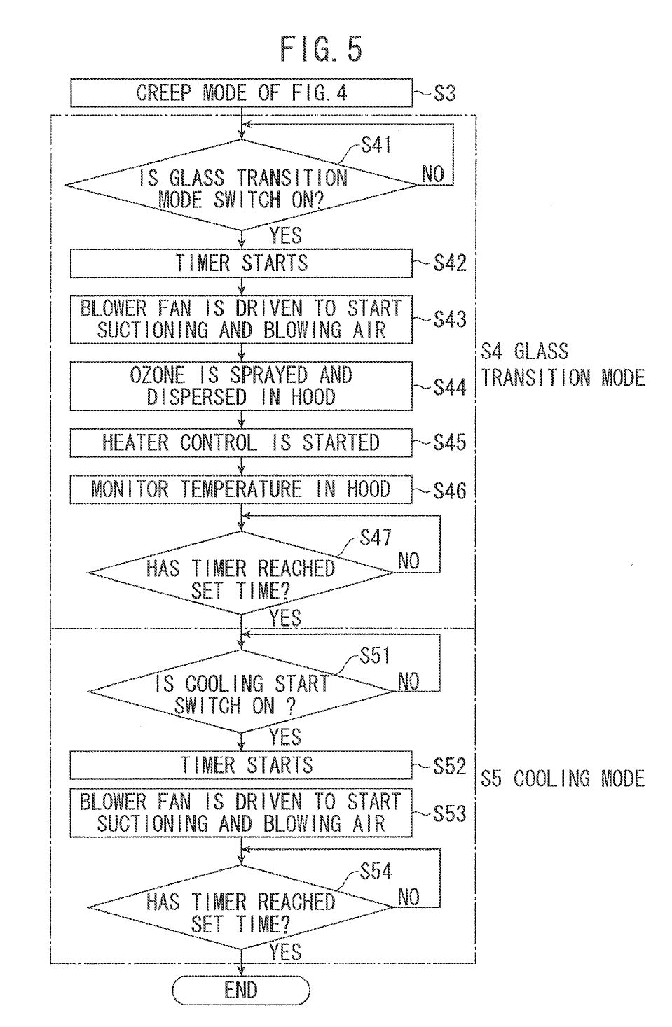

Simultaneously with the start of the timer in step S32, the blower 12 and the atmospheric air introducing device of the permanent wave treatment apparatus A are driven, whereby hot air is blown out from the hot air blowout port (step S33). To keep the temperature in the hood 2 constant, the CPU starts controlling the heater to bring the hot air to the temperature set in the ROM (step S34). The temperature sensor is used to monitor the temperature in the hood 2 and controls the temperature to the temperature set in the ROM (step S35). The CPU subsequently determines whether or not the timer has reached the time set in the ROM (step S36), and if it is determined that the set time has been reached, the procedure moves to glass transition mode S4 shown in FIG. 5.

After the practitioner connects the tube to the rod, the start switch for starting the glass transition will be operated, so it is essential to confirm that the tube is connected to the rod. As shown in FIG. 5, the CPU monitors whether or not the practitioner has switched on the switch for starting the glass transition (step S41). If it is determined that it has been switched on, the timer set in program setting mode S1 starts (step S42). Simultaneously with the start of the timer in step S42, the treatment apparatus A again drives the blower 12 and atmospheric air introducing device to blow out hot air and suctions the hot air through the rod around which hair is wound, to create a negative pressure in the rod (step S43).

Simultaneously with the start of the operations of step S43, the permanent wave treatment apparatus A discharges ozone from the ozone unit 3, with the ozone riding the hot air from the hot air blowout port of the blower 12 being evenly diffused within the hood 2 (step S44). In order to keep the temperature in the hood 2 constant, the CPU subsequently starts controlling the heater (step S45), uses the temperature sensor to monitor the temperature in the hood 2, and controls the temperature to the temperature set in the ROM (step S35). Continuing on, it is determined whether or not the timer has reached the time set in the ROM (step S47). If it is determined that the set time has been reached, the procedure moves to cooling mode S5.

At this stage, since the interior of the rod is already in a negative pressure state, ozone supplied into the hood 2 is suctioned via the hair, the ozone's own oxidizing action oxidizes the hair, the ascorbic acid salt contained in the chemical agent applied to the hair is oxidized to form dehydroascorbic acid salt which also oxidizes the hair, thereby recoupling the disulfide bonds of the hair.

When the procedure moves to cooling mode S5, the CPU monitors whether or not the practitioner has switched on the cooling start switch (step S51). If it is determined that it has been switched on, the timer starts at the setting set in program settings mode S1 (step S52). Simultaneously with the start of the operations in step S52, the permanent wave treatment apparatus A drives (heater power is interrupted) to blower 12 and the atmospheric air introducing device to suction cold air or blown air from the inside of the rod around which the hair are wound, thereby promoting the cooling of the hair (step S53).

Next, it is determined whether or not the timer has reached the time set in the ROM (step S54). If it is determined that the set time has been reached, the series of controls by the control section is terminated, and the practitioner opens the hood 2 and removes the rod from the hair, completing the operation.

The above-described permanent wave treatment method also promotes creep treatment after the chemical agent is blown onto the hair. Therefore, since there is no tube connected to the rod, there is no suctioning of the chemical agent from the rod around which the hair is wound. However, the present invention can also carry out the treatment of blowing the chemical agent onto the hair after the creep treatment and directly before the glass transition treatment is done.

In this case, since during the glass transition treatment the tube is connected to the rod, by spraying on the chemical agent just before carrying out the glass transition treatment and then creating the negative pressure state in the rod, the chemical agent in the form of a mist can be uniformly spread on the hair wound around the rod and efficiently adhered to the hair by the negative pressure in the rod. Also, the suction of the negative pressure state in the rod delivers hot air in the hood 2 onto the hair, resulting in a good finish.

When the negative pressure state is formed in the rod by connecting the tube to the rod from when the chemical agent is being sprayed, the chemical agent is spread uniformly over the entire hair and can be adhered to the hair with good efficiency. In this case too, the inside of the rod will also be in the negative pressure state in the next step, i.e. the creep treatment, the negative pressure state delivers hot air in the hood 2 onto the hair, resulting in a good finish.

### LIST OF REFERENCE SYMBOLS

A Permanent wave treatment apparatus A
1 Apparatus body
2 Hood
3 Ozone unit
4 Chemical agent mist unit
11 Housing
12 Blower
12a Suction port
13 Air-water separator
21 First hood
22 Second hood
31 Ozone blowout port
41 Chemical agent atomization kettle
42 Chemical agent spray member
43 Chemical agent mist fan
44 Chemical agent spray member drive motor

1. A permanent wave treatment method, comprising the steps of applying a first permanent wave agent having a reduction action to swell and soften hair wound around a rod, rinsing off the first permanent wave agent, applying to the hair one, two or more compounds selected from a group consisting of ascorbic acid, ascorbic acid derivative, and ascorbic acid salt, and an intermediate treatment agent, spraying ozone at the hair to oxidize the compound, and using the oxidized compound and the ozone to recouple disulfide bonds of the hair.

2. The permanent wave treatment method according to claim 1, wherein the intermediate treatment agent contains a compound whose concentration is 0.13 to 24 percent by weight, and an ozone atmosphere is formed containing not more than 1.0 ppm of the ozone.

3. The permanent wave treatment method according to claim 1, wherein, in the recoupling of the disulfide bonds of the hair, the compound is adhered to the hair wound around the rod and a moisture of the hair is maintained while heating is applied for a prescribed time, after which a negative pressure state is established inside the rod, and while using a blower for drying, ozone is sprayed towards the hair to envelop the hair in an ozone atmosphere.

4. The permanent wave treatment method according to claim 1, wherein the oxidized compound is dehydroascorbic acid or a dehydroascorbic acid salt.

5. The permanent wave treatment method according to claim 1, wherein a second permanent wave agent having an oxidizing action is applied to the hair in which disulfide bonds have been recoupled, to further recouple disulfide bonds.

6. The permanent wave treatment method according to claim 2, wherein, in the recoupling of the disulfide bonds of the hair, the compound is adhered to the hair wound around the rod and a moisture of the hair is maintained while heating is applied for a prescribed time, after which a negative pressure state is established inside the rod, and while using a blower for drying, ozone is sprayed towards the hair to envelop the hair in an ozone atmosphere.

7. The permanent wave treatment method according to claim 2, wherein the oxidized compound is dehydroascorbic acid or a dehydroascorbic acid salt.

8. The permanent wave treatment method according to claim 3, wherein the oxidized compound is dehydroascorbic acid or a dehydroascorbic acid salt.

9. The permanent wave treatment method according to claim 2, wherein a second permanent wave agent having an oxidizing action is applied to the hair in which disulfide bonds have been recoupled, to further recouple disulfide bonds.

10. The permanent wave treatment method according to claim 3, wherein a second permanent wave agent having an oxidizing action is applied to the hair in which disulfide bonds have been recoupled, to further recouple disulfide bonds.

11. The permanent wave treatment method according to claim 4, wherein a second permanent wave agent having an oxidizing action is applied to the hair in which disulfide bonds have been recoupled, to further recouple disulfide bonds.
